(19) United States
(12) Patent Application Publication
(10) Pub. No.: US 2021/0353546 A1
(43) Pub. Date: Nov. 18, 2021

(54) DUAL RELEASE PHARMACEUTICAL COMPOSITIONS COMPRISING THE COMBINATION OF A BETA-3 ADRENORECEPTOR AGONIST AND A MUSCARINIC RECEPTOR ANTAGONIST

(71) Applicant: Jubilant Pharma Holdings Inc., Yardley, PA (US)

(72) Inventors: Indranil Nandi, Yardley, PA (US); Tusharmouli Mukherjee, Yardley, PA (US)

(73) Assignee: Jubilant Pharma Holdings Inc., Yardley, PA (US)

(21) Appl. No.: 17/317,630

(22) Filed: May 11, 2021

Related U.S. Application Data
(60) Provisional application No. 63/023,753, filed on May 12, 2020.
(60) Provisional application No. 63/024,381, filed on May 13, 2020.

(51) Int. Cl. A61K 9/20; A61K 31/426; A61K 31/439; A61K 9/00; A61K 9/28

(52) U.S. Cl. CPC A61K 9/2086; A61K 31/426; A61K 31/439; A61K 9/2054; A61K 9/2013; A61K 9/2893; A61K 9/009; A61K 9/2866; A61K 9/284; A61K 9/2846; A61K 9/2027

(57) ABSTRACT

The present invention relates to a pharmaceutical composition comprising a combination of mirabegron and solifenacin or their pharmaceutically acceptable salts, wherein the composition comprises mini-tablets, multiparticulates, inlay tablets, or bilayer tablets. The prior art discloses restrictive formulation techniques and suggests complexity for preparing the combination in a single formulation to achieve the desired technical attributes. The test formulations are stable and exhibit desired pharmaceutical technical attributes. The invention also relates to the use of the pharmaceutical composition of the present invention in the treatment of various diseases like overactive bladder and other related therapeutic indications.

### FIELD OF THE INVENTION

The present invention relates to a stable pharmaceutical composition comprising a combination of solifenacin and mirabegron or their pharmaceutically acceptable salts, esters, solvates, polymorphs, enantiomers or mixtures thereof, and at least one pharmaceutically acceptable excipient, wherein mirabegron is in modified release form and solifenacin is in immediate release form. In particular. the present invention discloses a stable pharmaceutical composition wherein the composition is in the form of multiparticulates or granules or powder to be encapsulated in the capsule, dispensed into a sachet, or compressed to mini-tablets, inlay tablets, or bilayer tablets. The invention also provides a process for manufacturing such compositions and use of the said composition as a medicament in the treatment of various diseases like overactive bladder and other related therapeutic indications.

### BACKGROUND OF THE INVENTION

Mirabegron is an orally active beta-3 adrenoreceptor agonist approved for the treatment of overactive bladder. Mirabegron is chemically described as 2-(2-aminothiazol-4-yl)-N-[4-(2-{[(2R)-2-hydroxy2-phenylethyl] amino} ethyl) phenyl] acetamide. Mirabegron is practically insoluble in water and soluble in methanol and dimethyl sulfoxide. The half-life of mirabegron is approximately 50 hours and the bioavailability is affected by the presence of food in the Gastrointestinal Tract (GIT). To overcome the food effect issue, the commercially available formulation of mirabegron is in the form of a modified-release (MR) dosage form. The structural formula of mirabegron is as follows:

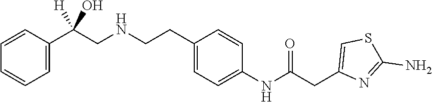 [SMILES: Nc1nc(CC(=O)Nc2ccc(CCNC[C@H](O)c3ccccc3)cc2)cs1]

Mirabegron is approved as extended release tablets of 25 and 50 mg strength under the brand name Betmiga® in Europe and Myrbetriq® in the United States of America (USA). Each Myrbetriq® extended release tablet, for oral administration, contains inactive ingredients like polyethylene oxide, polyethylene glycol, hydroxypropyl cellulose, butylated hydroxytoluene, magnesium stearate, and hypromellose.

Mirabegron and a process for its preparation are disclosed in U.S. Pat. No. 6,346,532 assigned to Astellas Pharma.

US Patent Publication No. 2017/0231965 assigned to Astellas Pharma discloses that the bioavailability of immediate release mirabegron is adversely impacted by the presence of food in the GIT. Thus, to prevent the food effect the marketed 25 mg and 50 mg tablet formulations of mirabegron were developed in the form of a modified-release (MR) tablet formulation based on an orally controlled absorption system (OCAS®) dosage form technology.

Solifenacin succinate is one of the muscarinic receptor antagonists indicated for the treatment of overactive bladder with associated problems such as increased urination frequency and urge urinary incontinence. Chemically, it is butanedioic acid, compounded with (1S)-(3R)-1-azabicyclo [2.2.2]oct-3-yl 3, 4-dihydro-1-phenyl-2(1H)-isoquinolinecarboxylate (1:1) having a molecular weight of 480.55.

Solifenacin succinate is represented by the following structural formula:

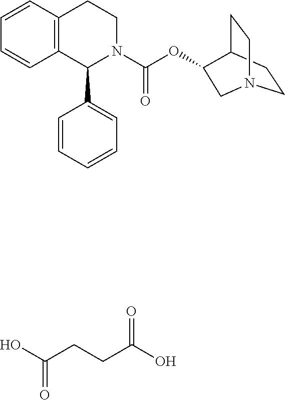 [SMILES: O=C(O[C@H]1CN2CCC1CC2)N1CCc2ccccc2[C@@H]1c1ccccc1 ; O=C(O)CCC(=O)O]

Solifenacin succinate is approved for oral administration as a tablet dosage form in the United States of America (USA) under the tradename VESIcare®. Each VESIcare® tablet contains lactose monohydrate, cornstarch, hypromellose, magnesium stearate, talc, polyethylene glycol 8000, and titanium dioxide with yellow ferric oxide or red ferric oxide.

Recently, the United States Food and Drug Administration (USFDA) approved a Supplementary New Drug Application (sNDA) for the use of mirabegron in combination with the muscarinic antagonist solifenacin succinate for the treatment of overactive bladder (OAB) with symptoms of urge urinary incontinence. The use of mirabegron extended release tablets in combination with solifenacin succinate is now part of the Myrbetriq® approved label or patient information leaflet, wherein both the drugs are administered separately as (a) 5 mg solifenacin succinate and 25 mg mirabegron, and (b) 5 mg solifenacin succinate and 50 mg mirabegron.

Various dosage forms comprising a combination of mirabegron and solifenacin succinate are known in U.S. Pat. No. 8,772,315, US Patent Publication No. 2015/0306090, and PCT Patent Publication No. WO2015129893 assigned to Astellas Pharma.

US Patent Publication No. 2019/0307696 assigned to Synthon BV discloses a multi-layer tablet comprising a controlled release part with mirabegron and an immediate release part wherein the immediate release formulation comprises solifenacin succinate and a water insoluble diluent.

The inventors of the instant application have determined that the combination product of solifenacin succinate and mirabegron has one or more of the following challenges: a) stability issues due to the generation of solifenacin succinate and mirabegron analogues, b) adverse change of dissolution rate of mirabegron during storage, and c) both drugs are known to have physical and chemical incompatibility.

The inventors of the present invention have developed novel combination formulations of mirabegron and solifenacin succinate in a single dosage form. The dosage forms are in the form of mini-tablets of mirabegron and solifenacin, to be filled into capsules, sachets, or stick packs and in bilayer tablet dosage form wherein mirabegron is in modified release form and solifenacin succinate is in immediate release form. The prepared dosage forms are stable and exhibit desired pharmaceutical technical attributes.

### SUMMARY OF THE INVENTION

It is an object of the present invention to develop a stable pharmaceutical composition comprising a combination of mirabegron and solifenacin or their pharmaceutically acceptable salts, wherein both the drugs are in a single dosage form having the same or different release profile.

Another object of the present invention is to develop a stable pharmaceutical composition comprising a combination of mirabegron and solifenacin or their pharmaceutically acceptable salts thereof, wherein the composition is in the form of mini-tablets to be encapsulated in a capsule or dispensed into a sachet or stick pack.

Another object of the present invention is to develop a stable pharmaceutical composition comprising a combination of mirabegron and solifenacin or their pharmaceutically acceptable salts thereof, wherein the composition is in the form of a bilayer tablet in which mirabegron is in modified release form and solifenacin succinate is in immediate release form, optionally separated by a coating or a non-functional layer.

Yet another object of the invention is to develop a stable pharmaceutical composition of the said combination, which exhibits desired formulations technical attributes like stability, dissolution, and assay.

### DESCRIPTION OF THE INVENTION

The present invention can be more readily understood by reading the following detailed description of the invention and study of the included examples.

As used herein, the term “composition”, as in pharmaceutical composition, is intended to encompass a drug product comprising an active or its pharmaceutically acceptable salt or derivative thereof, and the other inert ingredient(s) (pharmaceutically acceptable excipients). Such pharmaceutical compositions are synonymous with “formulation” and “dosage form”. Pharmaceutical compositions of the invention include, but are not limited to, mini-tablets, spherules, beadlets, granules, pellets, multiparticulates, microcapsules, and the like, filled in a capsule, a sachet, or a stick pack or compressed to tablets, wherein the composition is a fixed dose combination. Preferably, the pharmaceutical composition refers to mini-tablets using one or more different Active Pharmaceutical Ingredients (API), filled into capsules or sachets. Preferably, the pharmaceutical composition refers to powder, granules, multiparticulates, mini-tablets, and the like using one or more different APIs, filled into capsules or sachets; or powder, granules, multiparticulates compressed into bilayer tablets or inlay tablets.

Mini-tablets are convenient for use in pediatric and geriatric patients since they are easier to swallow than conventional tablets and capsules. In addition, they can be used to meet a full range of dissolution profiles, including delayed-release, controlled-release, extended release, and combination or dual-release profiles.

The term “mini-tablets” or “mini tablets” used herein refers to a plurality of micro-tablets, which are round, circular, disc like or cylindrical with a flat or convex upper side and lower side and with a diameter and height, which are preferably approximately equal and independent of one another, from 1.0 mm to 5.0 mm, preferably 1.5 mm to 2.5 mm. The minitablets as used herein refers to a composition comprising one or more APIs and at least one or more excipients selected from diluent, binder, disintegrant, lubricant, glidant, surfactant, wetting agent, solubilizer, stabilizer, cushioning agent, sweetener, flavoring agent, coloring agent, carrier, and one or more release controlling polymers, etc.

The term “multiparticulate” as used herein refers to a plurality of discrete or aggregated particles, pellets, powder, granules, beads, spheroids, spheres, microspheres, spherules, beadlets, multiparticulates, microcapsules, millispheres, and the like or mixture thereof, irrespective of their size, shape or morphology.

The term “excipient” means a pharmacologically inactive component such as a diluent, binder, disintegrant, antioxidant, lubricant, glidant, surfactant, wetting agent, solubilizer, stabilizer, anticaking agent, sweetener, flavoring agent, coloring agent, coating agent, carrier, and one or more rate-controlling polymers, etc. or the like. The excipients that are useful in preparing a pharmaceutical composition are generally safe, non-toxic, and are acceptable for veterinary as well as human pharmaceutical use. Reference to an excipient includes both one and more than one such excipient. Co-processed excipients are also covered under the scope of the present invention. Further, the excipient may be in the form of powders or in the form of a dispersion. Combinations of excipients performing the same function may be used to achieve desired formulation characteristics.

As used herein, the pharmaceutically acceptable salt(s) include, but are not limited to, maleic, citric, gluconic, lactic, malic, mandelic, cinnamic, citraconic, aspartic, stearic, palmitic, itaconic, glycolic, p-aminobenzoic, glutamic, benzene sulfonic and theophylline acetic acids, fumaric, benzoic, ascorbic, embonic, succinic, oxalic, bismethylenesalicylic, methanesulfonic, ethanedisulfonic, acetic, propionic, tartaric, salicylic, 8-halotheophyllines (e.g. 8-bromo-theophylline), hydrochloric, hydrobromic, sulfuric, sulfamic, phosphoric and nitric acids and the like.

As used herein, the term “about” means t approximately 20% of the indicated value, such that “about 10 percent” indicates approximately 08 to 12 percent. The term “w/w” as used herein refers to the total weight of the composition. Unless otherwise stated the weight percentages expressed herein are based on the final weight of the composition, the core, the coated core, mini-tablet, the multiparticulate, or the bilayer tablet.

“Single dosage form” or “unit dosage form” as used herein refers to a specific form of product that exists individually, and is available for a single administration and contains one or more APIs. Single dosage form herein refers to granules, powder, pellets, multiparticulates, mini-tablets filled into capsule and sachet; or granules, powder, pellets, multiparticulates compressed into tablets, bilayer tablet, and inlay tablets.

A “bilayer tablet” is a tablet produced by compaction of different granules in the form of various layers into a single tablet. It generally consists of parallel distinct layers with two or more APIs optionally along with functional or non-functional layers, to avoid incompatibilities between APIs by physical separation, and to enable the development of different drug release profiles (immediate release with extended release).

As used herein, the term “immediate release” refers to pharmaceutical compositions that release at least 60% or more of the active ingredient within a small period of time, typically less than 30 minutes or 45 minutes.

As used herein, the term “modified release” refers to pharmaceutical compositions that either release the active ingredient at a sustained or controlled release rate over a period of time such as for example at least 10%, 20%, and 30% released in 2 hours, 4 hours, 6 hours, 8 hours, 12 hours, 16 hours and 24 hours or release the active ingredient after a predetermined time.

As used herein, “dual release compositions” can combine the active ingredient in immediate release form with the additional active ingredient in a modified release form. For example, one or more extended release beads, granules, pellets, multiparticulates, mini-tablets, slugs, powder, spheres, etc. of one active ingredient can be combined with one or more immediate release beads, multiparticulates, powder, granules, pellets, slugs, spheres, mini-tablets, etc. of the other active ingredient. Alternatively, one active is in an extended release core coated with another active immediate release coat. In addition, the term includes inlay tablets, bilayer tablets, and multilayer tablets.

The term “sequential-release” as used herein, refers to a release of one or more active ingredients from a dosage form that comprises a first population and a second population, wherein the first population releases the active ingredient in delayed, sustained, controlled or extended release manner and the second population releases the active ingredient in immediate release manner or conversely.

The term “sachet” as used herein refers to any suitable container, foil, package, or bag to contain the composition. The sachet may be formed of any suitable material, including plastic, metal foil, paper, or a combination thereof. The sachet can be three-layered with sandwiched polyethylene terephthalate (PET)/aluminum/polyethylene layers or four-layered or more with the addition of more layers of PET/aluminum/polyethylene to provide robust protection to moisture-sensitive drugs or a stick pack. The sachet may be provided with any suitable means for opening thereof, including a perforated region or a nick in the edge of the sachet for ease of tearing. The sachet may be of any suitable size. The sachet is sealed using any appropriate method. Particularly, the sachet is disposable. The sachet can be a child-resistant container.

The term “stable” refers to the compositions of the present invention, wherein substantially no analogues or degradation product of any API is generated during storage of the dosage form, preferably for at least 3 months, more preferably for at least 6 months. The stability of the solid oral composition may be evaluated at “long-term” conditions of 25° C./60% RH (Relative Humidity), at “intermediate conditions” of 30° C./65% RH, and at “accelerated conditions” of 40° C./75% RH in the final container or pack measured as either the assay or drop in dissolution. Stability testing may be conducted according to the current guidelines set out by the ICH (International Council for Harmonisation) and USFDA (United States Food and Drug Administration).

A first aspect of the present invention relates to a pharmaceutical composition comprising a combination of mirabegron and solifenacin or their pharmaceutically acceptable salts, wherein the composition comprises a plurality of mini-tablets.

A second aspect of the present invention relates to a pharmaceutical composition comprising a combination of mirabegron and solifenacin comprising a plurality of mini-tablets, wherein mirabegron is in modified release form and solifenacin is in immediate release form.

A third aspect of the present invention relates to a pharmaceutical composition comprising a combination of mirabegron and solifenacin or their pharmaceutically acceptable salts, wherein the composition comprises: a) a first component comprising about 0.1% to about 80% by weight of mirabegron, about 1% to about 70% by weight of one or more release controlling polymers, and one or more pharmaceutically acceptable excipients; and b) a second component comprising about 0.1% to about 40% by weight of solifenacin and one or more pharmaceutically acceptable excipients, wherein the first component is in an extended release form with the weight ratio of drug to release controlling polymer comprises from 1:0.5 to 1:5 and the second component is in an immediate release form.

Another aspect of the present invention relates to a pharmaceutical composition comprising a combination of mirabegron and solifenacin or their pharmaceutically acceptable salts, wherein the first component is in the form of mini-tablets and the second component is in the form of granules, powder, coating, or mini-tablets.

Another aspect of the present invention relates to a pharmaceutical composition comprising a combination of mirabegron and solifenacin or their pharmaceutically acceptable salts, wherein solifenacin or its pharmaceutically acceptable salts preferably comprises solifenacin succinate. Another aspect of the present invention relates to a pharmaceutical composition comprising a combination of mirabegron and solifenacin or their pharmaceutically acceptable salts, wherein one or more release controlling polymers are hydrophobic polymers selected from cellulose derivatives, ethylcellulose, sodium alginate, carbomer, polyethylene glycol, sodium carboxymethyl cellulose, xanthan gum, guar gum, glyceryl behenate, locust bean gum, vinylpyrolidone vinyl acetate copolymer (PVP/VA) polymers, methacrylates, and polyvinyl alcohol.

A fourth aspect of the present invention relates to a pharmaceutical composition comprising a combination of mirabegron and solifenacin, wherein the composition comprises: a) a first component comprising about 5% to about 40% by weight of mirabegron, about 5% to about 60% by weight of one or more release controlling polymers, and one or more pharmaceutically acceptable excipients; b) a second component comprising about 0.5% to about 10% by weight of solifenacin and one or more pharmaceutically acceptable excipients, wherein an extended release component of the formulation exhibits a dissolution profile of more than 35% of total mirabegron being released in 3 hours, more than 50% of total mirabegron being released in 7 hours, and more than 60% of total mirabegron being released in 10 hours, when measured in a USP type II apparatus, in 900 mL of a USP buffer, pH 6.8 at 100 rpm. An immediate release component of the formulation exhibits at least 60% or more release of solifenacin in 30 minutes, when measured in a 500 ml of 0.1 N HCL using a USP apparatus I at 100 rpm at 37° C.

A fifth aspect of the present invention relates to a process for preparing mini-tablets comprising a combination of mirabegron and solifenacin or their pharmaceutically acceptable salts, wherein the process is selected from dry granulation, wet granulation, direct compression, drug layering, coating, hot-melt extrusion, extrusion spheronization or spray drying. Wet granulation is the preferred method for manufacturing the present invention.

A sixth aspect of the present invention relates to a pharmaceutical composition comprising a plurality of mini-tablets, wherein the mini-tablets comprise about 1% to about 70% of release controlling polymers based on the total weight of the composition.

A seventh aspect of the present invention relates to a pharmaceutical composition comprising a plurality of mini-tablets, wherein the mini-tablets are filled into a pharmaceutical grade capsule, sachet, or stick pack.

An eighth aspect of the present invention relates to a pharmaceutical composition comprising a plurality of mini-tablets, wherein each mini-tablet has a diameter of less than 5.0 mm.

A ninth aspect of the present invention relates to a pharmaceutical composition comprising a combination of mirabegron and solifenacin or their pharmaceutically acceptable salts, wherein the composition comprises, consists essentially of, or consists of:
a) an extended release mini-tablet comprising:
   i. about 0.1% to about 80% by weight of mirabegron,
   ii. about 1% to about 70% by weight of one or more release controlling polymers,
   iii. optionally one or more other pharmaceutically acceptable excipients;
b) optionally a seal coat over the mini-tablets; and
c) an immediate release outer coating layer comprising about 0.1% to about 40% by weight of solifenacin, one or more polymers, and one or more pharmaceutically acceptable excipients,
   wherein the weight ratio of drug to release controlling polymer comprises from 1:0.2 to 1:5.

A tenth aspect of the present invention relates to a stable pharmaceutical composition comprising a combination of mirabegron and solifenacin or their pharmaceutically acceptable salts, wherein the composition comprises: a) mini-tablets comprising about 5% to about 40% by weight of mirabegron, about 5% to about 60% by weight of one or more release controlling polymers and one or more pharmaceutically acceptable excipients, b) optionally a seal coat, and c) an outer coating layer comprising about 0.5% to about 10% by weight of solifenacin and one or more pharmaceutically acceptable excipients, wherein about 1 to about 20 coated mini-tablets are filled into capsules or sachets to form 25 mg/5 mg, 50 mg/5 mg, 25 mg/10 mg and 50 mg/10 mg of the combination wherein mirabegron is present in 25 mg and 50 mg and solifenacin is present in 5 mg and 10 mg, respectively.

An eleventh aspect of the present invention relates to a pharmaceutical composition comprising a combination of mirabegron and solifenacin or their pharmaceutically acceptable salts, wherein the composition comprises:
a) mini-tablets comprised of:
   i. about 5% to about 40% by weight of mirabegron,
   ii. about 5% to about 60% by weight of one or more release controlling polymers selected from cellulose derivatives, hydroxypropyl methylcellulose, hydroxypropyl cellulose, ethylcellulose, polyoxyethylene oxide, sodium alginate, carbomer, polyethylene glycol, sodium carboxymethyl cellulose, xanthan gum, guar gum, locust bean gum, vinylpyrrolidone vinyl acetate copolymer (PVP/VA) polymers, methacrylate polymers or copolymers, and polyvinyl alcohol,
   iii. optionally one or more other pharmaceutically acceptable excipients;
   iv. optionally a seal coat; and
b) an outer coating layer comprising about 0.5% to about 10% by weight of solifenacin, one or more polymers, and one or more pharmaceutically acceptable excipients,
   wherein one or more polymers used in the outer coating layer are selected from hydroxypropyl methylcellulose, ethylcellulose, polyethylene glycol, polyacrylic acid, polyvinyl alcohol, polyvinyl acetate, and polyvinylpyrrolidone.

A twelfth aspect of the present invention relates to a process for preparing a pharmaceutical mini-tablet, wherein the process comprises the steps of: a) blending mirabegron with diluent, one or more release controlling polymers, disintegrant, and optionally one or more other pharmaceutically acceptable excipients to obtain a blended mixture, b) granulating the blended mixture with a binder solution, c) drying the granulate obtained in step b), d) lubricating, and compressing the dried granules, and e) preparing an aqueous dispersion that comprises solifenacin and a polymer with one or more other pharmaceutically acceptable excipients, f) coating the mini-tablets of step d) with the dispersion of step e) to form the coated mini-tablets and drying the coated tablets.

A thirteenth aspect of the present invention relates to a pharmaceutical composition comprising a plurality of mini-tablets comprising a combination of mirabegron and solifenacin or their pharmaceutically acceptable salts, wherein the mini-tablet has a diameter of less than 5 mm and the solifenacin coat has a thickness ranging from 5 μm to 20 μm.

Another aspect of the present invention relates to a pharmaceutical composition comprising a plurality of mini-tablets comprising a combination of mirabegron and solifenacin or their pharmaceutically acceptable salts, wherein the solifenacin coat is present from about 1% to 10% by total weight of the coated mini-tablet.

In another aspect, pharmaceutically acceptable excipients are selected from the group comprising diluents, binders, disintegrants, antioxidants, lubricants, glidants, plasticizers, wetting agents, solubilizers, stabilizers, anticaking agents, antifoaming agents, alkaline agents, film-forming polymers, opacifiers, coloring agent, and surfactants.

Another aspect of the present invention relates to a bilayer tablet comprising a combination of mirabegron and solifenacin or their pharmaceutically acceptable salts, wherein the composition comprises: a) an extended release layer comprises about 0.1% to about 80% of mirabegron, about 1% to about 70% release controlling polymer, and one or more pharmaceutical acceptable excipients, wherein the layer is free of any permeation enhancer; b) an immediate release layer comprises solifenacin in an amount from about 0.1% to about 40% and one or more pharmaceutical acceptable excipients; and c) optionally a non-functional layer or film coat in between the first layer and the second layer, wherein the weight ratio between the first layer and the second layer is from about 1:10 to about 10:1.

Another aspect of the present invention relates to a bilayer tablet comprising a combination of mirabegron and solifenacin or their pharmaceutically acceptable salts, wherein the release controlling polymers in the extended release layer are selected from hydrophobic polymers like ethylcellulose, polyoxyethylene oxide, sodium alginate, carbomer, sodium carboxymethyl cellulose, glyceryl behenate, xanthan gum, guar gum, locust bean gum, vinylpyrolidone vinyl acetate copolymer (PVP/VA) polymers, methacrylates, polyvinyl alcohol and/or any mixtures thereof.

Another aspect of the present invention relates to a bilayer tablet comprising a combination of mirabegron and solifenacin or their pharmaceutically acceptable salts, wherein the composition comprises: a) an extended release layer comprises about 0.1% to about 80% of mirabegron, about 1% to about 70% release controlling polymer, and one or more pharmaceutical excipients, wherein the layer is free of any permeation enhancer; b) an immediate release layer comprises solifenacin in an amount from about 0.1% to about 40%; and c) optionally, a non-functional layer or film coat in between the first layer and the second layer, wherein the extended release component of formulation exhibits dissolution profile as more than 35% of total mirabegron is released in 3 hours, more than 50% of total mirabegron is released in 7 hours, and more than 60% of total mirabegron is released in 10 hours, when measured in a USP type II apparatus, in 900 mL of a USP buffer, pH 6.8 at 100 rpm and immediate release component exhibits at least 60% or more release of solifenacin in 30 minutes, when measured in a 500 ml of 0.1 N HCL using a USP apparatus I at 100 rpm at 37° C.

Another aspect of the present invention relates to a bilayer tablet comprising a combination of mirabegron and solifenacin or their pharmaceutically acceptable salts, wherein the bilayer tablet is compressed with a compression force from 2 to 30 kN.

Another aspect of the present invention relates to a bilayer tablet comprising a non-functional layer (i.e., layer without API) between the mirabegron and solifenacin layer to avoid the undesirable interactions of these incompatible API's. The non-functional layer can comprise excipients such as sugars, lactose, cellulose derivatives such as microcrystalline cellulose (MCC), hydroxypropylcellulose (HPMC) or phosphates derivatives such as dicalcium phosphate or hydrogenated castor oil or waxes or any other as known in the art.

Another aspect of the present invention relates to a bilayer tablet comprising a combination of mirabegron and solifenacin or their pharmaceutically acceptable salts, wherein release-controlling polymers in the extended release layer comprises about 20% to about 60% by weight of the bilayer tablet.

In another aspect of the present invention, the weight ratio of mirabegron to the release controlling polymers comprises from 1:0.2 to 1:2. In another aspect of the present invention, the weight ratio of mirabegron to the release controlling polymers comprises from 1:0.5 to 1:5. In another aspect of the present invention, the weight ratio of mirabegron to the release controlling polymers comprises from 1:3.1 to 1:5.

In another aspect of the present invention, the weight ratio of mirabegron to the release controlling polymers ranges from about 1:0.5:5 to about 1:5:0.5, preferably from about 1:0.5:3 to about 1:3:0.5, more preferably from about 1:0.5:2 to about 1:2:0.5, wherein the release controlling polymers are selected from hydroxypropyl methylcellulose, hydroxypropyl cellulose, ethylcellulose, polyoxyethylene oxide, sodium alginate, carbomer, polyethylene glycol, sodium carboxymethyl cellulose, hydrogenated castor oil, xanthan gum, guar gum, locust bean gum, vinylpyrrolidone vinyl acetate copolymer (PVP/VA) polymers, methacrylate polymers or copolymers, polyvinyl alcohol and/or any combination or mixture thereof.

Another aspect of the present invention relates to a pharmaceutical composition comprising a combination of mirabegron and solifenacin or their pharmaceutically acceptable salts, wherein extended release component of formulation exhibits dissolution profile as at least 10% of total mirabegron is released in 2 hours, at least 30% of total mirabegron is released in 8 hours, and at least 80% of total mirabegron is released in 12 hours, and immediate release component exhibits at least 60% or more release of solifenacin in 30 minutes.

Another aspect of the present invention relates to a pharmaceutical composition wherein mirabegron exhibits dissolution profile as more than 35% in 3 hours, more than 50% in 7 hours, and more than 60% in 10 hours, when measured in 900 ml phosphate buffer (pH 6.8) using a USP apparatus 1 (basket) at 100 rpm at 37° C. The solifenacin in the composition exhibits a dissolution profile as at least 60% or more in 30 minutes, when measured in a 500 ml of 0.1 N HCL using a USP apparatus I (Basket) at 100 rpm at 37° C.

Another aspect of the present invention relates to a pharmaceutical composition wherein solifenacin exhibits dissolution profile as at least 60% or more in 30 minutes when measured in 900 ml water using a USP apparatus 2 (paddles) at 50 rpm at 37° C.

Another aspect of the present invention relates to a pharmaceutical composition comprising a combination of mirabegron and solifenacin or their pharmaceutically acceptable salts, wherein the dose of mirabegron ranges from about 10 mg to about 100 mg and the dose of solifenacin ranges from about 0.1 mg to about 20 mg. Preferably, the dose of mirabegron is about 15 mg to about 60 mg and solifenacin is from about 1 mg to about 10 mg. More preferably, the dose of mirabegron is about 25 mg to about 50 mg and solifenacin is from about 5 mg to about 10 mg, preferably, the salt of solifenacin is solifenacin succinate.

Various sizes and shapes for the mini-tablets are contemplated within the scope of the present invention. In one embodiment, the mini-tablets may comprise a diameter of from about 1 mm to about 7.8 mm, preferably from about 1.5 mm to about 3.5 mm.

Various processes for producing the mini-tablets are contemplated herein. For example, a carrier may be sprayed with a suspension comprising mirabegron and an extended release polymer inside a fluidized bed granulator to form a granulated mixture. In addition, the suspension may also include other excipients. The granulated mixture can be milled and blended with other optional ingredients (e.g., lubricants and disintegrants). The blend is then compressed into extended release cores/mini-tablets/layer, and then a coating suspension comprising one or more water-soluble polymers and solifenacin succinate is applied to the cores/mini-tablets. The mini-tablet composition has less tendency to stick during coating, thereby allowing for higher spray rates and decreased coating time.

The diameter of the mini-tablet ranges between 3.0 to 6.0 mm. These mini-tablets are then filled into a hard gelatin capsule. Preferably, about 16, 12, 6, 3 mini-tablets are filled into a pharmaceutically acceptable capsule or sachet to form 25/5 mg and 50/5 mg strengths of the combination, respectively, wherein 25 mg and 50 mg corresponds to mirabegron and 5 mg corresponds to solifenacin, respectively. The thickness of the coating layers can be analyzed using Scanning Electron Microscopy (SEM).

In another aspect of the present invention, the compositions of the present invention are prepared by either dry granulation or direct compression, or wet granulation. In another embodiment, the invention relates to a process for the preparation of extended release dosage form comprising less than or equal to 80% by weight of mirabegron and one or more pharmaceutically acceptable excipients and a release controlling polymer. The process includes (i) granulating mirabegron, release controlling polymer, and diluent using binder solution, (ii) drying the granules of step (i), (iii) lubricating the dried granules of step (ii), and (iv) compressing the lubricated granules of step (iii) using 1.0 mm to 2.5 mm punches into mini-tablets.

In another embodiment, the invention relates to a process for the preparation of bilayer tablets comprising a combination of mirabegron and solifenacin or their pharmaceutically acceptable salts, wherein extended release layer is prepared by (i) granulating mirabegron, release controlling polymer, and diluent using binder solution, (ii) drying the granules of step (i), (iii) lubricating the dried granules of step (ii); and immediate release layer is prepared by (iv) granulating solifenacin and diluent using binder solution, (v) drying the granules of step (iv), (vi) lubricating the dried granules of step (v), compressing the granules of steps (iii, and (vi) into two separate layers.

In another embodiment of the invention, the compositions prepared as per the present invention have a hardness from about 1 to about 70 Newtons (N), and a friability of less than 2% when measured by USP (United States Pharmacopoeia) method. Preferably, the hardness of the tablet according to the present invention is between about 15 N to 40 N. Preferably, the friability of the tablet according to the present invention is not more than 1%. Several devices can be used to test tablet hardness such as a Monsanto tester, a Strong-Cobb tester, a Pfizer tester, an Erweka tester, a Schleuniger tester, etc. Friability can be determined using a Roche friabilator for 100 revolutions at 25 rpm (revolutions per minute). In another embodiment of the invention, there is provided a mini-tablet composition, which has a diameter of about 1.0 mm to about 5.0 mm, preferably, 1.5 mm to 2.5 mm. In another embodiment of the invention, there is provided a mini-tablet composition that has a Carr's index in the range of about 1-30%, preferably about 20% value, more preferably about 5% to 15% value, which indicates good flowability.

In another embodiment of the invention, there is provided a mini-tablet composition of mirabegron and solifenacin, wherein the composition has the assay in the range from 90% to 110% as measured by HPLC (High-Performance Liquid Chromatography) method using a suitable column.

In another embodiment of the invention, content uniformity of the mini-tablet composition is within acceptable limits and meets the requirements for dosage uniformity, in the acceptance value less than or equal to 15% as measured by a suitable technique such as HPLC (High-Performance Liquid Chromatography) method.

Another aspect of the present invention relates to a pharmaceutical composition comprising a combination of mirabegron and solifenacin or their pharmaceutically acceptable salts, having the desired bulk density and tapped density.

Another aspect of the present invention relates to a pharmaceutical composition comprising a combination of mirabegron and solifenacin or their pharmaceutically acceptable salts, wherein the composition is stable for at least 1 month in accelerated conditions.

Another aspect of the present invention relates to a pharmaceutical composition comprising a combination of mirabegron and solifenacin or their pharmaceutically acceptable salts, wherein undesirable analogues of mirabegron and solifenacin are within acceptable limits and further, there was no impact on the dissolution profile of both the drugs during stability.

Another aspect of the present invention relates to a pharmaceutical composition comprising a combination of mirabegron and solifenacin or their pharmaceutically acceptable salts, wherein there is no change in the polymorphic form of both the APIs.

Yet another aspect of the present invention relates to a pharmaceutical composition comprising a combination of mirabegron and solifenacin or their pharmaceutically acceptable salts, which is stable at 40° C. and 75%±5% relative humidity at least for a period of 3 months.

In yet another aspect, the present invention relates to a pharmaceutical composition comprising a combination of mirabegron and solifenacin or their pharmaceutically acceptable salts, used in the treatment of overactive bladder (OAB) with symptoms of urge urinary incontinence, urgency, and urinary frequency, and other diseases like metabolic diseases and adipose tissue-related diseases.

In another aspect, the present invention relates to a pharmaceutical composition, wherein said composition has no food effect when administered with food. In another aspect, the present invention relates to a pharmaceutical composition, wherein said composition has a minimal food effect when administered with food.

In another aspect of the present invention, there is provided a process for the preparation of mini-tablets or bilayer tablets by using conventional methods known in the art, including but not limited to, blending, mixing, dry granulation, wet granulation, direct compression, drug layering, coating, hot-melt extrusion, extrusion spheronization, spray drying, and spray coating techniques. Suitable solvents include aqueous or organic solvents. Preferable solvents include, but are not limited to, water, esters such as ethyl acetate, ketones such as acetone, alcohols such as methanol, ethanol, isopropanol, butanol, dichloromethane, chloroform, dimethylacetamide (DMA), dimethyl sulfoxide (DMSO), ether, diethyl ether and combinations thereof. Preferably, the solvent used during wet mass preparation is water or a hydroalcoholic solvent or mixture thereof.

In another aspect of the invention, wet granulation can be performed using a rapid mixer granulator, a fluid bed granulator, a planetary mixer, and the like; dry blending can be performed in V-blender or key blender; spheronization can be performed using Fuji Paudal spheronizer or by any other appropriate method known in the art.

Diluents or fillers or carriers are substances that usually provide bulk to the composition. Various useful fillers or diluents include, but are not limited to microcrystalline cellulose, silicified microcrystalline cellulose, calcium carbonate, calcium phosphate, dibasic anhydrous, calcium phosphate dibasic dihydrate, calcium phosphate tribasic, calcium sulphate, lactose, magnesium carbonate, magnesium oxide, maltodextrin, mannitol, sorbitol, starch, pregelatinized starch, sucrose, or mixtures thereof. The diluent may be present in an amount of about 5% w/w to about 80% w/w, preferably from about 10% w/w to about 70% w/w and more preferably from about 10% w/w to about 50% w/w.

Binders impart cohesiveness to formulation. Various useful binders include, but are not limited to hypromellose, acacia, alginic acid, carbomer, sodium carboxymethylcellulose, dextrin, ethylcellulose, gelatin, glucose, guar gum, hydroxypropylcellulose, maltose, methylcellulose, povidone, vinylpyrolidone vinyl acetate copolymer (PVP/VA) polymers, copovidone, starch, polyvinyl alcohol or polyethylene oxide, or mixtures thereof. The binder may be present in an amount of about 2% w/w to about 40% w/w, preferably from about 5% w/w to about 30% w/w and more preferably from about 5% w/w to about 20% w/w.

Glidants improve flowability and accuracy of dosing. Glidants used in the composition include, but are not limited to, tribasic calcium phosphate, calcium silicate, cellulose powdered, colloidal silicon dioxide, magnesium silicate, magnesium trisilicate, starch, and talc or mixtures thereof. The amount of glidant ranges from about 0.1% to about 5% w/w of the total composition.

The lubricants used in the present invention include calcium stearate, magnesium stearate, zinc stearate, light mineral oil, glyceryl behenate, polyethylene glycol, sodium stearyl fumurate, stearic acid, and talc. The lubricant may be present in an amount of about 0.1% w/w to about 2% w/w, preferably from about 0.5% w/w to about 1.5% w/w.

Surfactants contemplated in the present invention include, but are not limited to, anionic surfactants, amphoteric surfactants, non-ionic surfactants, and macromolecular surfactants. Suitable examples of surfactants include but are not limited to sodium lauryl sulphate, polysorbates (e.g. polysorbate 20, polysorbate 40, polysorbate 60, and polysorbate 80), macrogols, sodium cetyl stearyl sulphate, or sodium dioctyl sulphosuccinate, etc. The surfactant may be present in an amount of about 0.5% w/w to about 10% w/w, preferably from about 1% w/w to about 5% w/w and more preferably from about 0.5% w/w to about 2% w/w.

Various useful disintegrants and/or super-disintegrants include, but are not limited to, croscarmellose sodium, carboxymethyl cellulose sodium, carboxymethyl cellulose calcium, povidone, crospovidone, polacriilin potassium, sodium starch glycolate, alginic acid, sodium alginate, calcium phosphate, colloidal silicon dioxide, docusate sodium, guar gum, low substituted hydroxypropyl cellulose, magnesium aluminum silicate, methylcellulose, microcrystalline cellulose, silicified microcrystalline cellulose, starch, pre-gelatinized starch and/or combinations thereof. The disintegrant can be present in intra-granular or extra-granular part or in both (intra and extra granular) part of the composition. The amount of disintegrant according to the present invention ranges from about 0 to about 40% by weight of the composition. In an embodiment, the disintegrant according to the present invention is present in an amount of about 40% or less, 30% or less, e.g. 20% or less, 10% or less.

Various useful preservatives according to the present invention include, but are not limited to, parabens such as methylparaben, propylparaben, butylparaben or their salts, benzoic acid or its salts, sodium benzoate, potassium benzoate, calcium benzoate, methyl hydroxybenzoate, sodium metabisulphite, chlorhexidine, sodium citrate, butylated hydroxyl toluene (BHT), butylated hydroxyl anisole (BHA), tocopherol and the like. In particular, the preservative is selected from benzoic acid or its salts, butylated hydroxyl toluene (BHT), butylated hydroxyl anisole (BHA), and parabens. The preservative may be present in an amount of about 0.5% w/w to about 10% w/w, preferably from about 0.5% w/w to about 2% w/w.

The capsule shell can be gelatin-based or HPMC (hydroxypropyl methylcellulose or hypromellose)-based or PVA (Polyvinyl acetate)-based or cellulose ether film-based or starch-based. The size of the capsule may be selected to adequately contain the dose of the API(s). The capsule sizes according to the present invention ranges from 5 to 000 including sizes 000, 00, 0, 1, 2, 3, 4, and 5.

The final formulations according to the present invention may be coated or uncoated. For coating, additional excipients such as film-forming polymers, plasticizers, anti-adherents, and opacifiers are used.

Various water-soluble polymers are used to form a barrier/seal or film over the core. Examples include, but are not limited to, cellulose derivatives such as soluble alkyl- or hydroalkyl cellulose derivatives such as methylcellulose, hydroxymethyl cellulose, hydroxyethyl cellulose, vinylpyrrolidone vinyl acetate copolymer (PVP/VA) polymers, hydroxypropyl cellulose, hydroxymethylethyl cellulose, hydroxypropyl methylcellulose, sodium carboxymethyl cellulose, etc., polyacrylic acid, polyvinyl alcohol, polyvinyl acetate, polyvinylpyrrolidone, chitosan and derivatives thereof, shellac and derivatives thereof, waxes and fat substances. The water-soluble polymers may be present in an amount of about 1% w/w to about 80% w/w, preferably from about 5% w/w to about 70% w/w and more preferably from about 10% w/w to about 60% w/w. The films may contain additional adjuvants for coating such as plasticizers, polishing agents, colorants, pigments, antifoaming agents, opacifiers, anti-sticking agents, and the like.

Suitable acrylate and water-insoluble polymers suitable for use in the present invention include, but are not limited to, cellulose acetate phthalate (CAP), cellulose acetate trimellitate (CAT), hydroxypropylmethylcellulose phthalate (HPMCP), hydroxypropylmethylcellulose acetate succinate (HPMCAS), hydroxypropylcellulose acetate phthalate (HPCAP), hydroxypropylmethylcellulose acetate phthalate (HPMCAP), ethylcellulose (EC), polyvinyl acetate phthalate methylcellulose acetate phthalate (MCAP) and methacrylic acid copolymers or its derivatives. Acrylate polymers or methacrylic acid copolymers or its derivatives are selected from the group comprising different grades of Poly(butyl methacrylate, (2-dimethylaminoethyl) methacrylate, methyl methacrylate) 1:2:1, Poly(methacrylic acid, methyl methacrylate) 1:2, Poly(ethyl acrylate, methyl methacrylate, trimethylammonioethyl methacrylate chloride) 1:2:0.2, Poly(ethyl acrylate, methyl methacrylate, trimethylammonioethyl methacrylate chloride) 1:2:0.1, Poly(methacrylic acid, ethyl acrylate) 1:1.

Examples of a coating method include, for example, using a coating pan, or a fluidized bed.

Examples of the membrane-forming agents as used herein include, for example, one or more water-insoluble polymers or one or more water-soluble polymers. The membrane-forming agent is not particularly limited, so long as it is pharmaceutically acceptable and biocompatible. These membrane-forming agents may be added alone or as a combination thereof, in an appropriate amount(s).

In another embodiment of the present invention, release-controlling polymers are selected from hydrophobic polymers, water-soluble polymers, and gums. In yet another embodiment of the present invention, release controlling polymers are selected from hydroxyethyl cellulose, hydroxypropyl cellulose, sodium alginate, carbomer, polyethylene glycol, sodium carboxymethyl cellulose, xanthan gum, guar gum, locust bean gum, polyvinyl alcohol, vinylpyrrolidone vinyl acetate copolymer (PVP/VA) polymers, and hydroxypropyl methylcellulose. The preferred hydrophilic rate-controlling polymer that can be used in the present invention is hydroxypropyl cellulose and hydroxypropyl methylcellulose. The hydrophilic rate-controlling polymer comprises from about 1% w/w to about 80% w/w, preferably from about 5% w/w to about 70% w/w, and most preferably from 10% w/w to 60% w/w by weight of the composition. The hydrophilic polymer described above can also be used as a constituent of the coating layer.

The hydrogel-forming polymer may be selected from polyethylene oxide, hypromellose, hydroxypropyl cellulose, carboxymethyl cellulose sodium, hydroxyethyl cellulose, carboxyvinyl polymers, and combinations thereof. Examples of polyethylene oxide (also referred to as PEO) include product names, Polyox WSR-N-60K, PEO-27, PEO-18Z, PEO-15Z, PEO-8Z, Polyox WSR-N-12K, Polyox WSR-1105, Polyox WSR-308, Polyox WSR-303, Polyox WSR-N-750, Polyox WSR-N-80, Polyox WSR Coagulant, Polyox WSR-301, Polyox WSR-205, and Polyox WSR-N-10, PEO-4, PEO-3Z, PEO-2, PEO-1Z, PEO-1NF, 3NF, 8NF, 15NF, 18NF, and 27NF.

Examples of hypromellose (also referred as HPMC) include product name, Metolose 90SH50000, Metolose SB-4, TC-5RW, TC-5S, TC-5R, TC-5M, TC-5E, Metolose 60SH-50, Metolose 65SH-50, Metolose 90SH-100, Metolose 90SH-100SR, Metolose 65SH-400, Metolose 90SH-400, Metolose 65SH-1500, Metolose 60SH-4000, Metolose 65SH-4000, Metolose 90SH-4000, Metolose 90SH-4000SR, Metolose 90SH-15000, Metolose 90SH-15000SR, and Metolose 90SH-30000, Benecel MHPC, and E464.

Examples of hydroxypropyl cellulose (also referred to as HPC) include HPC-SSL, HPC-SL, HPC-L, HPC-M, and HPC-H, Klucel HPC various grades, methocel K100LV, K4M, K15M, K100M CR, hydroxypropyl ether, E463, hyprolose, Klucel, Nisso HPC, oxypropylated cellulose, 2-hydroxypropyl ether; 2-hydroxypropyl ether (low substituted) cellulose, hyprolose, low-substituted; L-HPC, and oxypropylated cellulose.

Examples of carboxyvinyl polymers include acrypol, acritamer, acrylic acid polymer, carbomera, Carbopol, carboxy polymethylene, polyacrylic acid, carboxyvinyl polymer, Pemulen, Tego Carbomer, Carbopol 71G, Carbopol 981, Carbopol 934, and Carbopol 934P.

Hydrophobic release controlling polymers that can be used according to the present invention are selected from the group comprising of cellulose ether such as ethylcellulose, cellulose acetate, polyvinyl acetate, methacrylic acid esters neutral polymer, polyvinyl alcohol-maleic anhydride copolymers, hydroxypropyl methylcellulose phthalate, EUDRAGIT® EPO, EUDRAGIT® E100, EUDRAGIT® E 12,5, EUDRAGIT® L 12,5, EUDRAGIT® FS 30 D, EUDRAGIT® RSPO, EUDRAGIT® RLPO, EUDRAGIT® RL30D, EUDRAGIT® RL12,5, EUDRAGIT® S 100, hydrogenated castor oil, glyceryl behenate, waxes and the like. Combinations of two or more Eudragit grades in any ratio can be used to control the drug release in the present invention. Even the commercially available dispersion of film formers namely, EUDRAGIT® L-30D, EUDRAGIT® NE 30D, AQUACOAT® ECD-30, SURELEASE® E-7, EUDRAGIT® RS 30D, EUDRAGIT® RL 30D, etc. may be used to provide extended release composition.

In another embodiment, the present invention includes particle size of free drug particulate form of mirabegron and solifenacin or their pharmaceutically acceptable salts, wherein particle diameter at 90% cumulative volume (d₉₀) is less than about 100 μm, preferably less than 50 μm. Particle diameter at X % cumulative particle size reduction can be performed by techniques including but not limited to fluid energy milling, ball milling, colloid milling, roller milling, hammer milling, and the like. Particle size and particle size distribution can be measured by techniques such as Laser light scattering (e.g. Malvern Light Scattering), Coulter counter, microscopy, and the like.

In yet another embodiment of the invention, the pharmaceutical composition of mirabegron and solifenacin or its pharmaceutically acceptable salts thereof is packed in a packaging material selected from the group comprising of a foil, a pouch, blister, a sachet, stick pack, bottle, container or another suitable package as known in the pharmaceutical art.

The pharmaceutical oral dosage form prepared according to the present invention can be subjected to in vitro dissolution evaluation according to Test 711 “Dissolution” in the United States Pharmacopoeia 37, United States Pharmacopoeial Convention, Inc., Rockville, Md., 2014 (“USP”) to determine the rate at which the active substance is released from the dosage form, and the content of the active substance can be determined in solution by high-performance liquid chromatography. When comparing the test and reference products, dissolution profiles should be compared using a similarity factor (f₂). The similarity factor is a logarithmic reciprocal square root transformation of the sum of squared error and is a measurement of the similarity in the percent (%) of dissolution between the two curves.

f₂=50·log {[1+(1/n)Σₜ₌₁ⁿ(Rₜ−Tₜ)²]^{-0.5}·100}

Two dissolution profiles are considered similar when the f₂ value is equal to or greater than 50.

Having described the invention with reference to certain preferred embodiments, other embodiments will become apparent to one skilled in the art from consideration of the specification. The invention is further defined by reference to the following examples describing in detail the method for the preparation and testing of the said pharmaceutical composition. It will be apparent to those skilled in the art that many modifications, both to materials and methods, may be practiced without departing from the scope of the invention. The following examples are set out to illustrate the invention and do not limit the scope of the present invention.

### Examples

The following non-limiting examples are intended to further illustrate certain preferred embodiments of the invention. They are however, not intended to be limiting to the scope of the present invention.

Table 1 represents three formulations (Examples 1-3) wherein mirabegron is in the extended release core (i.e., mini-tablet or multiparticulate), optionally coated with a seal coat and an outer immediate release coating containing solifenacin succinate and prepared by using the quantitative formula as given in the following examples.

TABLE 1
Qualitative and quantitative formula Examples 1-3

| | Mirabegron Core (Quantities in % w/w) | | |
|---|---|---|---|
| Components | Example 1 | Example 2 | Example 3 |
| Mirabegron | 5-40 | 1-50 | 10-30 |
| Hydroxy Propyl Cellulose | 0-50 | 0-50 | 0-40 |
| Hydroxypropylmethylcellulose | 1-30 | — | 1-50 |
| Polyethylene oxide | — | 0-50 | 0-50 |
| Polyethylene glycol | 0-20 | 0-20 | 0-20 |
| Ethyl cellulose | — | — | 0-20 |
| Dibasic Calcium Phosphate | 0-50 | 0-50 | 0-50 |
| Povidone/Pregelatinized | 0-10 | 0-10 | 0-10 |
| starch/HPMC/HPC | | | |
| Crospovidone/Croscarmellose | 0-10 | 0-10 | 0-10 |
| sodium/Sodium starch glycolate | | | |
| Magnesium Stearate/calcium | 0-2 | 0-2 | 0-2 |
| sterate/Silicon dioxide/Silicified | | | |
| MCC | | | |
| Purified Water | q.s. | q.s. | q.s. |

Optional Seal coat

| | | | |
|---|---|---|---|
| Hypromellose | 1-20 | 1-20 | 1-20 |
| Polyvinyl alcohol | 0-20 | 0-20 | 0-20 |
| Talc | 1-10 | 1-10 | 1-10 |
| Triethyl Citrate | 1-5 | 1-5 | 1-5 |
| Sodium lauryl sulfate | 0-2 | 0-2 | 0-2 |
| Purified Water/solvents | q.s. | q.s. | q.s. |

Solifenacin Drug Coating

| | | | |
|---|---|---|---|
| Solifenacin succinate | 0.1-40 | 0.1-10 | 1-20 |
| Hypromellose | 0-20 | 0-20 | 0-20 |
| Triethyl citrate | 0-4 | 0-4 | 0-4 |
| Glycerol monostearate | 0-5 | 0-5 | 0-5 |
| Macrogol 400 | 0-10 | 0-10 | 0-10 |
| Butylated hydroxy toluene | 0-2 | 0-2 | 0-2 |

Preferred methods of manufacture include: wet granulation, dry granulation, direct compression, and extrusion-spheronization.

The wet granulation process includes weighing and mixing of the drug, release controlling polymer, and diluents. Adding binder solution to prepare the damp mass, screening the wet mass into pellets or granules, drying of moist granules, and sizing the granules by screening. Finally lubricating the sized granules using a lubricant and compressing the granules into mini-tablets.

The direct compression process comprises pre-milling of formulation ingredients (active drug substance and excipients), mixing of active drug substance with the powdered excipients (including the lubricant), and compressing of the mixed powders into tablets/mini-tablets. The dry granulation process includes weighing formulation ingredients, mixing of formulation ingredients, compression of mixed powders into slugs, milling and sieving of slugs, mixing with disintegrant and lubricant, and finally compressing the granules into tablets/mini-tablets.

Compressed tablets were optionally seal-coated and the solifenacin drug layer was applied by preparing a drug dispersion along with binder and polymers. The prepared compressed tablets encapsulating or filling the coated mini-tablets into capsule, sachet, or stick pack.

Table 2 represents three formulations (Examples 4-6) wherein mirabegron is in the extended release core (i.e., mini-tablet or pellet or multiparticulate), optionally coated with a seal coat and solifenacin, the second component of the formulation is in immediate release form in the form of powder, granules, pellets, multiparticulates, and mini-tablets.

TABLE 2
Qualitative and quantitative formula Examples 4-6

| | Mirabegron Mini-tablets (Quantities | | |
|---|---|---|---|
| | in % w/w) | | |
| Components | Example 4 | Example 5 | Example 6 |
| Mirabegron | 5-40 | 10-60 | 1-50 |
| Hydroxy propyl cellulose | 0-50 | 0-50 | 0-50 |
| Hydroxypropylmethyl | 1-50 | — | 1-50 |
| cellulose | | | |
| Polyethylene oxide | — | 1-50 | 0-50 |
| Polyethylene glycol | 0-20 | 0-20 | 0-20 |
| Ethyl cellulose | — | — | 0-50 |
| Dibasic Calcium Phosphate | 0-50 | 0-50 | 0-50 |
| Povidone/Pregelatinized | 0-10 | 0-10 | 0-10 |
| starch/HPMC/HPC | | | |
| Crospovidone/Croscarmellose | 0-10 | 0-10 | 0-10 |
| sodium/Sodium starch | | | |
| glycolate | | | |
| Magnesium Stearate/calcium | 0-2 | 0-2 | 0-2 |
| sterate/Silicon dioxide | | | |
| Butylated hydroxy toluene | 0-2 | 0-2 | 0-2 |
| Purified Water | q.s. | q.s. | q.s. |

Optional film coat

| | | | |
|---|---|---|---|
| Hydroxypropyl cellulose | 1-20 | 1-20 | 1-20 |
| Polyvinylalcohol | 0-20 | 0-20 | 0-20 |
| Methacrylate polymer | 0-20 | 0-20 | 0-20 |
| Talc | 1-10 | 1-10 | 1-10 |
| Triethyl Citrate | 1-5 | 1-5 | 1-5 |
| Sodium lauryl sulfate | 0-2 | 0-2 | 0-2 |
| Purified Water/solvents | q.s. | q.s. | q.s. |

Solifenacin Mini-tablets

| | | | |
|---|---|---|---|
| Solifenacin succinate | 0.1-40 | 0.1-10 | 1-20 |
| Hypromellose | 0-20 | 0-20 | 0-20 |
| Lactose monohydrate | 5-50 | 5-50 | 5-50 |
| Povidone/Pregelatinized | 0-10 | 0-10 | 0-10 |
| starch/corn starch/HPMC | | | |
| Crospovidone/Croscarmellose | 0-10 | 0-10 | 0-10 |
| sodium/Sodium starch | | | |
| glycolate | | | |
| Magnesium Stearate | 0-2 | 0-2 | 0-2 |

Preferred methods of manufacture are: wet granulation, dry granulation, direct compression, and extrusion-spheronization. The detailed process is discussed above. The compressed mini-tablets were encapsulated into a capsule or filled into a sachet or stick pack.

Table 3 represents nine formulations (Examples 7-15) wherein mirabegron is in the extended release core (i.e., mini-tablet or pellet or multiparticulate) and the second component of the formulation is in immediate release form in the form of powder, granules, pellets, multiparticulates, and mini-tablets.

### Manufacturing Process:

First Component: Mirabegron Extended Release Tablets:

Mirabegron, release controlling polymer, cornstarch, and magnesium stearate were co-sifted through a mesh. The mixture was blended to form a homogenous blend and compressed using a 3.00 mm punch to mini-tablets. For the wet granulation manufacturing process as described above for Examples 1-3, was used.

Second Component: Solifenacin Succinate Tablets:

Solifenacin succinate, lactose monohydrate, cornstarch, hypromellose were co-sifted through a suitable mesh. The blend was granulated using purified water. The wet granules were dried at 50° C.±10° C., to achieve the LOD: NMT (not more than) 3.0%. Dried granules were milled and then sifted through a suitable mesh. Extragranular part: Cornstarch was sifted through the suitable mesh and then blended with the sifted granules for 25 minutes. Afterwards, magnesium stearate was blended with the material in a blender. The lubricated blend was compressed using a 3.00 mm punch to mini-tablets.

Table 4-6 represents the technical data of mini-tablets such as physical parameters, assay and dissolution of formulations covered in Examples 7-9. Table 7 represents three bilayer tablet formulations (Examples 16-18) wherein a bilayer tablet comprises mirabegron in the extended release layer, optionally a non-functional layer, and an immediate release layer comprising solifenacin. The bilayer tablet formulations of Examples 16-18 exhibited desirable technical attributes.

TABLE 3
Qualitative and Quantitative formula examples 7-15

| | | | Example | Example | Example | Example | Example | Example | Example | Example | Example |
|---|---|---|---|---|---|---|---|---|---|---|---|
| | | | 7 | 8 | 9 | 10 | 11 | 12 | 13 | 14 | 15 |
| S. no. | Ingredients | Function | mg/unit dosage form | | | | | | | | |
| Mirabegron Extended Release | | | Mini-tablets | | | Granules/powder | | | Pellets | | |
| 1 | Mirabegron | Active | 25.00 | 25.00 | 25.00 | 25.00 | 25.00 | 25.00 | 25.00 | 25.00 | 25.00 |
| 2 | Ethyl Cellulose | Release | 15.00 | 75.00 | 125.00 | — | 50.00 | — | — | 50.00 | — |
| 3 | Glyceryl behenate | Controlling | — | — | — | 75.00 | — | — | 75.00 | — | — |
| 4 | Eudragit RSPO | Polymer | — | — | — | — | — | 25.00 | — | — | 25.00 |
| 5 | Eudragit RLPO | | — | — | — | — | — | 30.00 | — | — | 30.00 |
| 6 | Corn starch/pregelatinized | Diluent | 9.50 | 5.50 | 5.50 | 9.50 | 5.50 | 5.50 | 9.50 | 5.50 | 5.50 |
| | starch | | | | | | | | | | |
| 7 | Butylated Hydroxytoluene | Anti-oxidant | — | — | — | — | 0.20 | — | 0.20 | 0.10 | — |
| 8 | Magnesium stearate | Lubricant | 0.50 | 0.50 | 0.50 | 0.50 | 0.70 | 0.55 | 0.65 | 0.50 | 0.70 |
| 9 | Purified water | | — | — | q.s | — | q.s. | q.s | q.s | q.s. | q.s |

| | | | | | | | | | |
|---|---|---|---|---|---|---|---|---|---|
| Total (Mirabegron ER mini-tab/Capsule) | 50.00 | 106.00 | 156.00 | 108.00 | 81.4 | 86.05 | 86.00 | 81.1 | 86.2 |

Solifenacin Immediate Release mini-Tablets or granules or powder /Capsules
Intragranular portion

| | | | | | | | | | | | |
|---|---|---|---|---|---|---|---|---|---|---|---|
| 1 | Solifenacin Succinate | Active | 5.00 | 5.00 | 5.00 | 5.00 | 5.00 | 5.00 | 5.00 | 5.00 | 5.00 |
| 2 | Lactose Monohydrate | Diluent | 50.00 | 50.00 | 50.00 | 50.00 | 50.00 | 50.00 | 50.00 | 50.00 | 50.00 |
| 3 | Corn-starch | Disintegrant | 11.00 | 11.00 | 11.00 | 11.00 | 11.00 | 11.00 | 11.00 | 11.00 | 11.00 |
| 4 | Hypromellose | Binder | 4.50 | 4.50 | 4.50 | 4.50 | 4.50 | 4.50 | 4.50 | 4.50 | 4.50 |
| 5 | Purified water | Granulating | q.s | q.s | q.s | q.s | q.s | q.s | q.s | q.s | q.s |
| | | solvent | | | | | | | | | |

Extragranular portion

| | | | | | | | | | | | |
|---|---|---|---|---|---|---|---|---|---|---|---|
| 6 | Corn starch | Disintegrant | 4.00 | 4.00 | 4.00 | 4.00 | 4.00 | 4.00 | 4.00 | 4.00 | 4.00 |
| 7 | Magnesium stearate | Lubricant | 0.50 | 0.50 | 0.50 | 0.50 | 0.50 | 0.50 | 0.50 | 0.50 | 0.50 |

| | | | | | | | | | |
|---|---|---|---|---|---|---|---|---|---|
| Total (Solifenacin Succinate mini-tabs or | 75.00 | 75.00 | 75.00 | 75.00 | 75.00 | 75.00 | 75.00 | 75.00 | 75.00 |
| granules or powder/Capsule) | | | | | | | | | |
| Total Weight of dosage form | 125 | 181 | 231 | 183 | 156.4 | 161.05 | 161.00 | 156.1 | 161.2 |

TABLE 4
is a continuation of table 3, which represents various parameters of the final dosage form

| | | Example 7 | | Example 8 | | Example 9 | |
|---|---|---|---|---|---|---|---|
| | | | Number | Mini- | | Mini- | Number |
| | | Mini-tab | of mini- | tab | Number of | tab | of mini- |
| S. | | weight | tab/ | weight | mini-tab/ | weight | tab/ |
| no. | Ingredients | (mg) | Capsule | (mg) | Capsule | (mg) | Capsule |
| 1 | Solifenacin Succinate | 14.6 | 5 | 14.6 | 5 | 14.6 | 5 |
| | Tablet | | | | | | |
| 2 | Mirabegron Extended | 16.9 | 3 | 15.14 | 3 | 17.3 | 3 |
| | Release Tablets | | | | | | |

TABLE 5
represents the physical parameters of the mini-tablets

| | Solifenacin | | | |
|---|---|---|---|---|
| Physical Parameters | succinate mini- | Mirabegron ER mini-tablets | | |
| of (Mini-tablets) | tablets | Example 7 | Example 8 | Example 9 |
| Number of Mini | 3 Nos | 3 Nos | 7 Nos | 9 Nos |
| Tablets | | | | |
| Diameter (mm) | 3.0 mm | 3.0 mm | 3.0 mm | 3.0 mm |
| Thickness (mm) | 1.95 mm | 2.39 mm | 2.64 mm | 2.64 mm |
| Hardness (N) | 10 N | 38 N | 30 N | 38 N |

TABLE 6
represents the dissolution results of Solifenacin succinate and
Mirabegron Capsules

| | | | |
|---|---|---|---|
| Test Parameter | Example 7 | Example 8 | Example 9 |

Assay (% w/w)

| | | | |
|---|---|---|---|
| Solifenacin Succinate | 98.0 | 101.4 | 101.4 |
| Mirabegron | 101.2 | 101.0 | 101.4 |

Solifenacin succinate: % Drug release in 0.1 N HCL/500 mL/Basket/
100 rpm

| | | | |
|---|---|---|---|
| 10 min | 85 | 90 | 90 |
| 15 min | 97 | 98 | 98 |
| 20 min | 98 | 98 | 98 |
| 30 min | 99 | 98 | 97 |
| 45 min | 99 | 97 | 97 |
| 60 min | 98 | 97 | 96 |

Mirabegron: % Drug release in pH 6.8 Phosphate buffer/900 mL/
Basket/100 rpm

| | |
|---|---|
| 3 hrs | More than 35% |
| 7 hrs | More than 50% |
| 10 hrs | More than 60% |

TABLE 7
Qualitative and quantitative formula Examples 16-18

| | | |
|---|---|---|
| Example 16 | Example 17 | Example 18 |

| | | | |
|---|---|---|---|
| S.no. | Ingredients | Function | mg/unit dosage form |

Mirabegron Extended Release (ER) layer

| | | | | | |
|---|---|---|---|---|---|
| 1 | Mirabegron | Active | 25.00 | 25.00 | 25.00 |
| 2 | Ethyl Cellulose | Release | 75.00 | — | — |
| 3 | Glyceryl behenate | Controlling | — | 60.00 | — |
| 4 | Eudragit RSPO | Polymer | — | — | 35.00 |
| 5 | Eudragit RLPO | | — | — | 30.00 |
| 6 | Corn starch/pregelatinized | Diluent | 9.50 | 5.50 | 5.50 |
| | starch | | | | |
| 7 | Butylated Hydroxytoluene | Anti-oxidant | — | — | — |
| 8 | Magnesium stearate | Lubricant | 0.50 | 0.50 | 0.50 |
| 9 | Purified water | | — | — | q.s |

| | | | |
|---|---|---|---|
| Total (Mirabegron ER layer) | 110.00 | 91.00 | 96.00 |

Optional non-functional layer

| | | | | | |
|---|---|---|---|---|---|
| 1 | Lactose | Inert material | 40.00 | — | — |
| 2 | Microcrystalline cellulose | | — | 50 | — |
| 3 | Hydrogenated castor oil | | — | — | 35 |
| | Purified water | Granulating | q.s. | q.s. | q.s. |
| | | solvent | | | |

Solifenacin Immediate Release (IR) Layer
Intragranular portion

| | | | | | |
|---|---|---|---|---|---|
| 1 | Solifenacin Succinate | Active | 5.00 | 5.00 | 5.00 |
| 2 | Lactose Monohydrate | Diluent | 50.00 | 50.00 | 50.00 |
| 3 | Corn-starch | Disintegrant | 11.00 | 11.00 | 11.00 |
| 4 | Hypromellose | Binder | 4.50 | 4.50 | 4.50 |
| 5 | Purified water | Granulating | q.s | q.s | q.s |
| | | solvent | | | |

Extragranular portion

| | | | | | |
|---|---|---|---|---|---|
| 6 | Corn starch | Disintegrant | 4.00 | 4.00 | 4.00 |
| 7 | Magnesium stearate | Lubricant | 0.50 | — | 0.50 |
| 8 | Calcium stearate | Lubricant | — | 0.50 | — |

| | | | |
|---|---|---|---|
| Total (Solifenacin Succinate IR layer) | 75.00 | 75.00 | 75.00 |
| Total Weight of dosage form | 225 | 216 | 206 |

What is claimed:

1. A pharmaceutical composition comprising a combination of mirabegron and solifenacin or their pharmaceutically acceptable salts, wherein the composition comprises:
a) a first component comprising about 0.1% to about 80% by weight of mirabegron, about 1% to about 70% by weight of one or more release controlling polymers, and one or more pharmaceutically acceptable excipients;
b) a second component comprising about 0.1% to about 40% by weight of solifenacin and one or more pharmaceutically acceptable excipients,
wherein the first component is in an extended release form with the weight ratio of drug to release controlling polymer from 1:0.5 to 1:5 and the second component is in an immediate release form.

2. The pharmaceutical composition according to claim 1, wherein the first component is in the form of mini-tablets and the second component is in the form of granules, powder, coating, or mini-tablets.

3. The pharmaceutical composition according to claim 1, wherein the composition comprises:
a) the first component comprises about 5% to about 40% by weight of mirabegron, about 5% to about 60% by weight of one or more release controlling polymers, and one or more pharmaceutically acceptable excipients;
b) the second component comprises about 0.5% to about 10% by weight of solifenacin and one or more pharmaceutically acceptable excipients,
wherein an extended release component of the composition exhibits a dissolution profile of more than 35% of total mirabegron released in 3 hours, more than 50% of total mirabegron released in 7 hours, and more than 60% of total mirabegron released in 10 hours, when measured in a USP type II apparatus, in 900 mL of a USP buffer, pH 6.8 at 100 rpm and an immediate release component of the composition exhibits at least 60% or more release of solifenacin in 30 minutes, when measured in a 500 ml of 0.1 N HCL using a USP apparatus I at 100 rpm at 37° C.

4. The pharmaceutical composition according to claim 1, wherein solifenacin or its pharmaceutically acceptable salts comprises solifenacin succinate.

5. The pharmaceutical composition according to claim 1, wherein the one or more release controlling polymers are hydrophobic polymers selected from cellulose derivatives, ethylcellulose, sodium alginate, carbomer, polyethylene glycol, sodium carboxymethyl cellulose, xanthan gum, guar gum, glyceryl behenate, locust bean gum, vinylpyrolidone vinyl acetate copolymer (PVP/VA) polymers, methacrylates, and polyvinyl alcohol.

6. The pharmaceutical composition according to claim 1, wherein the one or more pharmaceutically acceptable excipients are selected from diluents, binders, disintegrants, antioxidant, lubricants, glidants, plasticizer, wetting agent, solubilizer, stabilizer, anticaking agent, antifoaming agent, alkaline agent, film-forming polymer, opacifiers, coloring agent, and surfactant.

7. A process for preparing a pharmaceutical composition according to claim 1, wherein the process is selected from dry granulation, wet granulation, direct compression, drug layering, coating, hot-melt extrusion, extrusion spheronization, or spray drying.

8. The pharmaceutical composition according to claim 2, wherein the mini-tablets have a diameter less than 5.0 mm.

9. The pharmaceutical composition according to claim 2, wherein about 1 to about 20 mini-tablets are filled into capsules or sachets to form 25 mg/5 mg, 50 mg/5 mg, 25 mg/10 mg, and 50 mg/10 mg of the combination, wherein mirabegron is present in 25 mg and 50 mg and solifenacin is present in 5 mg and 10 mg, respectively.

10. The pharmaceutical composition according to claim 2, wherein mini-tablets, granules, and powder are filled into a pharmaceutically acceptable capsule or sachet or stick pack.

11. The pharmaceutical composition according to 1, wherein said composition has no food effect when administered with food.

12. A method of treating urinary urgency, urinary frequency, and/or urge urinary incontinence associated with overactive bladder in a patient, wherein the method comprises administering the pharmaceutical composition according to claim 1 to an individual in need thereof.

13. A pharmaceutical composition comprising a combination of mirabegron and solifenacin or their pharmaceutically acceptable salts thereof, wherein the composition comprises:
a) an extended release mini-tablet comprising:
i. about 0.1% to about 80% by weight of mirabegron,
ii. about 1% to about 70% by weight of one or more release controlling polymers,
iii. optionally one or more other pharmaceutically acceptable excipients;
iv. optionally a seal coat over the mini-tablets; and
b) an immediate release outer coating layer comprising about 0.1% to about 40% by weight of solifenacin, one or more polymers, and one or more pharmaceutically acceptable excipients,
wherein the weight ratio of drug to release controlling polymer ranges from 1:0.2 to 1:5.

14. The pharmaceutical composition according to claim 13, wherein the mini-tablet has a diameter of less than 5 mm and the solifenacin coat has a thickness ranging from 5 μm to 20 μm.

15. The pharmaceutical composition as according to claim 13, wherein the process for preparing the mini-tablets comprises: (a) blending mirabegron with diluent, one or more release controlling polymers, disintegrant and optionally one or more other pharmaceutically acceptable excipients to obtain a blended mixture, (b) granulating the blended mixture with a binder solution, (c) drying the granulate obtained in step (b), (d) lubricating and compressing the dried granules to form mini-tablets, and (e) preparing an aqueous dispersion that comprises solifenacin or its pharmaceutically acceptable salts and a polymer with one or more other pharmaceutically acceptable excipients and (f) coating the mini-tablets of step (d) with the dispersion of step (e) to form the coated mini-tablets, and (g) drying the coated tablets.

16. A bilayer tablet comprising:
a) an extended release layer comprises about 0.1% to about 80% of mirabegron, about 1% to about 70% release controlling polymer, and one or more pharmaceutical excipients, wherein the layer is free of any permeation enhancer and
b) an immediate release layer comprises solifenacin or its pharmaceutically acceptable salts in an amount from about 0.1% to about 40%
c) optionally a non-functional layer or film coat positioned between the first layer and the second layer,
wherein the weight ratio between the first layer and the second layer is from about 1:10 to about 10:1.

17. The pharmaceutical composition according to claim 16, wherein the weight ratio of drug to release controlling polymer ranges from 1:0.5 to 1:5.

18. A bilayer tablet according to claim 16, wherein the bilayer tablet is compressed with a compression force between 2 to 30 kN.

19. The pharmaceutical composition according to claim 16, wherein the composition comprises:
a) an extended release layer comprising about 5% to about 40% of mirabegron, about 5% to about 60% release controlling polymer, and one or more pharmaceutical excipients, wherein the extended release layer is free of any permeation enhancer;
b) an immediate release layer comprising solifenacin or its pharmaceutically acceptable salts in an amount from about 0.5% to about 10%; and
c) optionally a non-functional layer or film coat positioned between the first layer and the second layer,
wherein extended release component of formulation exhibits a dissolution profile of more than 35% of total mirabegron released in 3 hours, more than 50% of total mirabegron released in 7 hours, and more than 60% of total mirabegron released in 10 hours, when measured in a USP type II apparatus, in 900 mL of a USP buffer, pH 6.8 at 100 rpm, and the immediate release component exhibits at least 60% or more release of solifenacin in 30 minutes, when measured in 500 ml of 0.1 N HCL using a USP apparatus I at 100 rpm at 37° C.

20. A bilayer tablet according to claim 16, wherein the amount of the release-controlling polymer in the extended release layer comprises about 20% to about 60% by weight of the bilayer tablet.
